(19) United States
(12) Patent Application Publication
(10) Pub. No.: US 2008/0214442 A1
(43) Pub. Date: Sep. 4, 2008

(54) Anti-Viral Effect of an Extract of Ganoderma Lucidum

(76) Inventors: Alice Yu, La Jolla, CA (US); Chi-Huey Wong, Rancho Santa Fe, CA (US); Jia Tsrong Jan, Taipei (TW); Y.-S. Edmond Cheng, Palo Alto, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/859,729

(22) Filed: Sep. 21, 2007

Related U.S. Application Data
(60) Provisional application No. 60/846,675, filed on Sep. 21, 2006.

(51) Int. Cl. A61K 38/16; C07K 14/00; A61P 31/12

(52) U.S. Cl. 514/8; 530/395

(57) ABSTRACT

Compositions and methods for the treatment viral infections in organisms, particularly flu virus. The methods and compositions of the disclosure involve the administration to mammals and immune cells of a fucose-containing glycoprotein fraction from Reishi. An extract of G. lucidum induces immune responses that are shown to induce increase survivability in mice injected with lethal does of influenza virus. The effect is increased when the G. lucidum extract is dosed prior to injection of the lethal dose of influenza virus.

### RELATED APPLICATION

This application claims the benefit of and priority to U.S. Provisional Application Ser. No. 60/846,675, filed Sep. 21, 2006.

### BACKGROUND

This disclosure relates to compounds and methods for treating influenza virus in organisms with extracts of Ganoderma lucidum and Ganoderma tsugae, otherwise known as “Reishi” and/or (for purposes of this document) (“F3”) provides medicinally active extracts and fractions, and methods of preparing the same, from components of

### SUMMARY

Compositions and methods for the treatment viral infections in organisms, particularly flu virus. The methods and compositions of the disclosure involve the administration to mammals and immune cells of a fucose-containing glycoprotein fraction from Ganoderma lucidum. The extract of G. lucidum induces immune responses that are shown to induce increase survivability in mice injected with lethal does of influenza virus. The effect is increased when the G. lucidum extract is dosed prior to injection of the lethal does of influenza virus.

According to a feature of the present disclosure, a composition is disclosed comprising a fucose-containing glycoprotein extract of Reishi wherein the extract is used to treat an organism having a virus.

According to a feature of the present disclosure, a composition is disclosed comprising at least a fucose-containing glycoprotein fraction of Reishi, wherein at least the fucose-containing glycoprotein fraction is used for the treatment of virus infections in an organism.

According to a feature of the present disclosure, a method is disclosed comprising administering to an organism a pharmacologically effective amount of a fucose-containing glycoprotein extract of Reishi to treat virus infections in an organism.

According to a feature of the present disclosure, a kit is disclosed comprising at least a fucose-containing glycoprotein extract of Reishi.

### DRAWINGS

The above-mentioned features and objects of the present disclosure will become more apparent with reference to the following description taken in conjunction with the accompanying drawings wherein like reference numerals denote like elements and in which:

FIG. 1 is a graph of an embodiment illustrating the survival rate of mice injected with a lethal dose of H1N1 virus when treated with F3;

FIG. 2 is a graph of an embodiment illustrating the survival rate of mice injected with a lethal dose of H1N1 virus when treated with F3;

FIG. 3 is a graph of an embodiment illustrating the survival rate of mice injected with a lethal dose of H1N1 virus and various concentrations of F3;

FIGS. 4A and 4B is a graph of an embodiment illustrating the survival rate of mice injected with a lethal dose of H1N1 virus and various concentrations of F3;

FIG. 5 is a graph of an embodiment illustrating the survival rate of mice injected with a lethal dose of H1N1 virus and F3 obtained from various sources or F3 fragments;

FIG. 6 is a graph of an embodiment illustrating the survival rate of mice injected with a lethal dose of H5N1 virus and F3;

FIG. 7 is a graph of an embodiment illustrating the survival rate of mice injected with a lethal dose of H5N1 virus and F3;

FIG. 8 are graphs of embodiments illustrating the serum concentrations of cytokines and chemokines at injection of F3 in mice;

FIG. 9 are graphs of embodiments illustrating the effect of F3 on various cell-types 72 hours after injection;

FIG. 10 is a graph of an embodiment illustrating the relative levels of granzyme B 72 hours after injection of F3;

FIG. 11 are graphs of embodiments illustrating the effect of F3 on various cell-types 72 hours after injection of F3 and a lethal dose of H1N1; and

FIG. 12 are graphs of an embodiment illustrating the effect of F3 on the ability of a virus to replicate in vivo.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

The bioactive polysaccharide fraction of G. lucidum is referred to as “Reishi-F3,” “SF3,” “Reishi polysaccharides-F3,” or “EORP” and is described in WO 2006/044616, incorporated herein by reference in its entirety. F3 is a fucose-containing glycoprotein fraction. The present disclosure also hereby incorporates by reference U.S. Utility application Ser. No. 11/549,215 filed on 13 Oct. 2006, as if fully disclosed herein.

Botanical drugs could open up an unique opportunity for discovery or synthesis of safer and more cost-effective drugs. Reishi a popular traditional Chinese medicinal mushroom, has been used during the past two millennia as a home remedy for maintaining and improving health and for preventing and treating disease (1,2). The regular consumption of Reishi in the form of tea or mushroom powder was believed to preserve the human vitality and to promote longevity (1,3). In addition, numerous reports demonstrated that Reishi extensively modulates immune functions both in vivo and in vitro. Reishi has been used to treat various human diseases such as allergy, arthritis, bronchitis, gastric ulcer, hyperglycemia, hypertension, chronic hepatitis, hepatopathy, insomnia, nephritis, neurasthenia, scleroderma, inflammation, and cancer (1).

Reishi has been shown to be an anti-tumor agent. The potential clinical values has attracted intense interest to study its function and acting mechanism for pharmacological opportunity. A number of studies demonstrate that the anti-tumor activity of polysaccharide fraction of Reishi occurs not through direct induction of cell apoptosis or inhibition of proliferation, but mainly through the immunoenhancing activities (13,14).

The present inventors discovered that Reishi, in addition to its anti-tumor effects, is also an anti-influenza agent based on its ability to enhance the host's defense system. In one aspect, this disclosure provides a method for treating or preventing a viral infection in a mammal, the method comprising administering to the mammal the F3 extract of Ganoderma lucidum, or subfractions thereof that comprise fucose-containing glycoprotein(s).

Until recently, the immunomodulating activities of Reishi polysaccharide was demonstrated to through Toll-like receptor (TLR 2 and TLR4)-mediated signaling pathways, which in turn, activate the host immune response for B cell proliferation and cytokine production (10,15). However, more functions have been reported including promoting the function of antigen-presenting cells, mononuclear phygocyte system, humoral immunity and cellular immunity, and activation of the expression of and interleukin IL-1, IL-6, IL-12, IFN-γ, TNF-α, GM-CSF, G-CSF in splenocyte (16) induction of IL-1, IL-6 and TNF-α from activated macrophages. The above results obtained by various methodologies demonstrated that the immunomodulating effects of G. lucidum were extensive, diverse, and occur on many levels.

According to one aspect, a fucose-containing glycoproteic fraction of an extract of Reishi, is disclosed. The phrase “fucose-containing glycoprotein fraction” refers to a constituent part of the extract that includes at least one of a polysaccharide and a glycopeptide comprising fucose residues.

The term “glycoprotein” or “glycopeptide” refers to a protein of any length and dimensions with covalently attached sugar units, either bonded via the OH group of serine or threonine O glycosylated) or through the amide NH₂ of asparagine (N-glycosylated) or portions thereof and/or proteins that contain oligosaccharide chains (glycans) covalently attached to their polypeptide backbones.

The term “polysaccharide” refers to a polymers of any length and dimensions comprising monosaccharide residues linked glycosidically in branched or unbranched chains.

“Administering” means oral, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection and infusion as well as co-administered as a component of any medical device or object to be inserted (temporarily or permanently) into the body.

The term “extract” refers to a concentrated preparation obtained by removing active constituents from a given substance; when the active constituents are included in a solvent, the removal of the active constituents can be performed by evaporating all or nearly all the solvent and adjusting the residual mass or powder to a prescribed standard; extracts are usually prepared in three forms, semiliquid or of syrupy consistency, pilular or solid and as dry powder. The phrase “Ganoderma lucidum” refers to fungus Ganoderma lucidum or Reishi, any tissue, part or fraction therefrom or any preparation thereof including homogenates, suspensions, filtrates, filtration residues, and solutions. The term “preparation” refers to a composition processed, manufactured, or compounded starting from a given substance, the term “concentrated preparation” refers to a preparation with an increased ratio of the mass or volume of active constituents to the mass or volume of the non-active constituents or to the mass volume of the entire composition, compared with the same ratio in the given substance. The term “fraction” refers to one of the separable constituents of a substance.

The fucose-containing glycoprotein fraction of Ganoderma lucidum constitutes an active constituent of the extract of Ganoderma lucidum, in view of the extraction procedures described below. The activities and properties of the fucose-containing glycoprotein fraction as well as methods and compositions using or including the fucose-containing glycoprotein fraction shall not be limited in scope and application by the examples, compositions, and procedures described herein.

In some embodiments, the fucose-containing glycoprotein fraction is included in a fraction of Ganoderma lucidum (herein also denominated F3, Fraction 3, EORP, GL(PS)_Wu, or Wu) showing a light absorbance of about 1.8 O.D. at 625 nm identified and isolated from a water-soluble extract of Ganoderma lucidum (crude Reishi extract) by experimental procedures described below.

Fraction 3 includes a fucose-containing glycoprotein fraction, which comprises terminal fucose residues. The phrase “terminal fucose residues” identifies fucose residues of a chain of sugars located in a region proximate to a free end of a chain of sugars. The fucose-containing glycoprotein fraction of Fraction 3, also includes fucose residues bound with α1,2-fucosidic linkages and α3,4-fucosidic linkages.

In addition to fucose residue, fucose-containing glycoprotein fraction of Fraction 3 can also comprise glucose mannose, N-acetylglucosamine, xylose, and rhamnose (see in part Table I).

The fucose-containing glycoprotein fraction of Fraction 3 can also include an amino acidic component. The amino acidic component of Fraction 3, however, can be significantly modified without impairing the activities associated with the fucose-containing glycoprotein fraction of Fraction 3.

Fraction F3 can be obtained by a process comprising: homogenizing a plant tissue of Ganoderma lucidum or providing an homogenized plant tissue from Ganoderma lucidum; extracting the homogenized plant tissue of Ganoderma lucidum; and filtering the extracted homogenized plant tissue to form one or more fractions, the fractions comprising a saccharide component having fucose residues. The fractions formed in the above procedures can also be treated with protease.

The term “extracting” refers to any suitable procedure or protocol to provide an extract starting from a given substance; determination of such protocols can be accomplished by those skilled in the art depending upon a variety of variables, including the substance and the active constituents to be removed from the substance; exemplary procedures include treatment based on different solubility of the constituents of the substance in different solvents. Extracting the homogenized plant tissue can be performed by any suitable procedure or protocol to provide an extract of Ganoderma lucidum including a fucose-containing glycoprotein or fucose-containing polysaccharide constituents from the homogenized plant tissue, such as described below. For example, a suitable procedure includes treating the homogenized plant tissue with aqueous alkaline solution, for example 0.1 N NaOH, for a predetermined time to form a crude extract.

“Gel filtration” means separation of proteins, peptides, and oligonucleotides on the basis of size. Molecules move through a bed of porous beads, diffusing into the beads to greater or lesser degrees. Smaller molecules diffuse further into the pores of the beads and therefore move through the bed more slowly, while larger molecules enter less or not at all and thus move through the bed more quickly. Both molecular weight and three dimensional shape contribute to the degree of retention. Gel Filtration Chromatography may be used for analysis of molecular size, for separations of components in a mixture, or for salt removal or buffer exchange from a preparation of macromolecules.

Reishi” means the name for one form of the mushroom Ganoderma lucidum, and its close relative Ganoderma tsugae.

“Hematopoietic Cells” means blood forming stem cells. T cells and B cells arise from these cells.

The term “filtering” or “filtration” refers to any suitable procedure to separate a constituent of a substance, such as an active constituent, from other constituents of the substance, such as impurities; determination of such protocols can be accomplished by those skilled in the art depending upon a variety of variables, including the substance, the active constituents and the inactive constituents of the substance; exemplary filtration procedures include dialysis and gel filtration chromatography. Filtering the extracted homogenized plant tissue can be performed by subjecting the crude extract to filtration, such as gel filtration chromatography e.g. using a Sephacryl S-500 column, and eluting with an aqueous solution to form one or more fractions. In one embodiment the aqueous solution is buffered at about pH 7.0, for example a Tris buffer solution.

Specific embodiments of the above-mentioned process to obtain Fraction 3 described in detail below.

According to embodiments, the fucose-containing glycoprotein fraction is included in fractions of Fraction F3 (herein also collectively named Subfractions), herein identified as F3G1, F3G2, F3G3, and the F3G2 sub-fractions F3G2H1 and F3G2H2. The Subfractions are isolated from Fraction 3. Hence, the term “fucose-containing glycoprotein” refers not only to Fraction F3, but also to any Subfractions thereof, and any combination of F3 with the Subfractions, or any combination of the Subfractions.

The different Subfractions can be identified by the respective ability to absorb light. F3G1 shows a light absorbance of about 0.4 O.D. at 480 nm, F3G2 shows a light absorbance of about 0.1 O.D. at 480 nm; F3G2H1 shows a light absorbance of about 0.10 O.D. at 480 nm and F3G2H2 shows a light absorbance of about 0.5 O.D. at 480 nm.

F3 and the Subfractions are also herein collectively named Fractions.

The fucose-containing glycoprotein fraction comprised in the Subfractions can also include, in addition to the fucose residues, other sugars such as glucose and mannose, galactose, N-acetylglucosamine, and xylose (see in particular Table IV).

The Subfractions F3G1, FG2 and F3G3 can be obtained by partitioning Fraction 3. The term “partitioning” refers to any suitable procedure or protocol to divide a substance in two or more constituents thereof; determination of such protocols can be accomplished by those skilled in the art depending upon a variety of variables, including the substance, and the constituents to be partitioned. Partitioning Fraction 3 can be performed by filtering Fraction 3, (for example with an anion exchanger such as Diaion-WA30 anion exchanger or by gel filtration chromatography, e.g. on a TSK HW-75 column), and isolating the Subfractions F3G1, F3G2, and F3G3 from the filtered Fraction 3, (for example by elution with an alkaline solution, including for example at least one of NaCl).

“Cytokine” means any of a group of proteins and peptides that are used in organisms as signaling compounds. These chemical signals are similar to hormones and neurotransmitters and are used to allow one cell to communicate with another.

F3G2 subfractions F3G2H1 ad F3G2H2 can be obtained by subjecting F3G2 to further partitioning. For example, partitioning of F3G2 can be performed by filtering Sub-fraction F3G2 e.g. by gel filtration chromatography e.g. on a TSK HW-75 column, and isolating the Subfractions F3G2H1 and F3G2H2 from the filtered Subfraction F3G2 e.g., by eluting the filtered Subfraction F3G2 with an aqueous solution.

According to embodiments, a method to mediate a biological event associated with activated expression of a cytokine in a mammalian cell, is disclosed. The phrase “biological event” refers to an occurrence being of or relating to biology or to life and living processes. The term “mediate” used interchangeably with “regulate” and “modulate” refers to specifically controlling, or influencing an item identified thereafter, the item including a molecule, pathway, event or function, wherein “mediate” can include regulation by activation, stimulation, inhibition, alteration or modification of such molecule, pathway event or function.

The term “express” or “expression” of a cytokine refers to a process by which the gene's coded information is converted into the cytokine. The term “activate” refers to initiating or enhancing of a process such as expression of a cytokine, or to converting a compound, such as protein nucleic acid lipid, ion or other compound, from an inactive into an active form or into a different compound, the active form or different compound having a particular biological action. The term “cytokines” refers to proteins or biological factors that are released by cells, such as normal macrophages, fibroblasts, keratinocytes, and a variety of transformed cell lines, have specific effects on cell-cell interaction, communication, and behavior of other cells, which participate in regulating immunological and inflammatory processes and can contribute to repair processes and to the regulation of normal cell growth and differentiation; cytokines include interleukins, lymphokines and several related signaling molecules such as TNF-α and interferons.

The phrase “mammalian cell” indicates a cell of mammalian origin, in particular a human or murine, which can be located inside or outside the mammal. The mammal cells can be a human or a murine cell, such as T lymphocytes, human and murine macrophage and murine spleen cells.

The cytokine can be IL-1, IL6, IFN-γ, TNF-α IL-12, GM-CSF, G-CSF or M-CSF, and the mammalian cell can be a human or a mouse cell, such as T lymphocytes, human and murine macrophages and monocytes, and murine spleen cells. In certain specific embodiments, the cytokine is IL-1, and the mammalian cell is a macrophage or a monocyte.

The method to mediate a biological event associated with activated expression of a cytokine in a mammalian cell comprises administering to the mammalian cell an effective amount of the fucose-containing glycoprotein fraction.

The term “effective amount” of a compound is at least the minimum amount of the compound that is necessary to minimally achieve the desired effect. An effective amount of fucose-containing glycoprotein fraction for use in a given method can be readily determined by one skilled in the art without undue experimentation, depending upon the particular circumstances encountered (e.g. concentrations, cell type and number, etc.) upon reading of the present disclosure and in particular the Examples section.

The term “administering” refers to any process or protocol suitable to put a compound, and in particular the fucose-containing glycoprotein fraction, in contact with the cell, wherein the term “contact” or the phrase “put in contact” mean to place the compound and in particular the fucose-containing glycoprotein fraction and the cell, in a mutual spatial relationship such that a biological interaction between the compound and the cell is feasible; the phrase “biological interaction” refers to the process by which a compound and in particular the fucose-containing glycoprotein fraction controls, influences or otherwise affects the normal functioning or survival of the cell; determination of such protocols can be accomplished by those skilled in the art depending upon a variety of variables, including the type of cell, whether the contact occurs in vitro, in vivo, or ex vivo. Acceptable protocols to administer the fucose-containing glycoprotein fraction include individual dose size, number of doses, frequency of dose administration, and mode of administration, such as topical administration, local administration, or oral administration in vivo, incubation and assays in vitro, or ex vivo administration, e.g., to isolated hematopoietic cells, which can be identified by a person skilled in the art upon reading of the present disclosure and, in particular, the Examples section.

In particular, the effective amount of fucose-containing glycoprotein fraction to mediate a biological event, and suitable modes of administration identifiable by a person skilled in the art in view of the biological event to be mediated and the cell where the cytokine expression is activated upon reading of the present disclosure, and in particular the Examples section.

In some embodiments the biological event is hematopoiesis, the mammalian cell is a hematopoietic cell and the cytokines are GM-CSF, G-CSF, and M-CSF. The term “hematopoiesis” refers to the formation of blood cells in a living body, (especially in the bone marrow of mammals). The term “hematopoietic cells” refers to a cell involved in the hematopoietic process, such as a B cell, a macrophage, a dendritic cell, and a natural killer cell.

In some embodiments, the biological event is hematopoiesis, the fucose-containing glycoprotein fraction is included in F3, F3G2, F3G2H1 or F3G2H1, possibly in combination with F3G1 and the effective amount to mediate hematopoiesis and suitable modes of administration can be identified by a person skilled in the art upon reading of the present disclosure.

In embodiments where the fucose-containing glycoprotein fraction is included in F3, the effective amount to mediate hematopoiesis and suitable modes of administration, are identifiable by a person skilled in the art upon reading of the present disclosure.

### Preparation and Analysis of Reishi Extracts F1, F2, F3, F4, and F5

Crude Reishi extract (prepared via alkaline extraction (0.1 N NaOH), neutralization and ethanol precipitation) was obtained from Pharmanex Co. (CA, USA). Twenty-eight mg of the crude extract were dissolved in 2 mL of Tris buffer (pH 7.0, 0.1 N) and centrifuged to remove the insoluble materials (7 mg). The supernatant was purified by gel filtration chromatography using a Sephacryl S-500 column (100×1.6 cm) with 0.1 N Tris buffer (pH 7.0) as the eluent. The flow rate was set at 0.5 mL/min, and the elute (7.5 mL per tube) was collected. Five fractions were collected (fractions 1-5), each dialyzed to remove excessive salt and lyophilized to give 1.0 mg, 6.2 mg, 5.3 mg, 2.1 mg, and less than 1 mg, respectively.

The main fraction having a light absorbance of about 1.8 at O.D. 625 was designated as Fraction 3. After the chromatography, the crude extract and each of the isolated fraction were subjected to anthrone analysis (Somani et al 1987; Jarmyn 1975; Halhoul and I. Kleinberg 1972) to detect sugar components.

### Sugar Composition Analysis: Anthrone Colorimetric Method

Each 1.5 mL of anthrone (9,10-dihydro-9-oxoanthracene) solution (0.2 g anthrone dissolved in 100 mL of concd sulfuric acid) in a series of test tubes immersed in an ice water bath was carefully overlayed with 1.5 mL of sample

(20-40 μg/mL of -glucose or equivalent). After all additions had been made, the tubes were shaken rapidly and then replaced in an ice water bath. The tubes were heated for 5 min in a boiling water bath and then cooled; the optical densities were read within an hour at 625 nm against distilled water. Standards, reagent blanks and unknowns were run in triplicate because of likely contamination by other carbohydrate sources. Calculations made on the basis that the optical densities are directly proportional to the carbohydrate concentration.

### Sugar Composition Analysis—TMS Method

For monosaccharide analysis, the polysaccharide extracts/fractions were methanolyzed with 0.5 M methanolic-HCl (Supelco) at 80° C. for 16 h, re-N-acetylated with 500 μL of methanol, 10 μL of pyridine, and 50 μL of acetic anhydride, and then treated with the Sylon HTP® trimethylsilylating reagent (Supelco) for 20 min at room temperature, dried and redissolved in hexane. GC-MS analysis of the trimethylsilylated derivatives was carried out using a Hewlett-Packard (HP) Gas Chromatograph 6890 connected to a HP 5973 Mass Selective Detector. Samples were dissolved in hexane prior to splitless injection into a HP-5MS fused silica capillary column (30 m×0.25 mm I.D., HP). The column head pressure was maintained at around 8.2 psi to give a constant flow rate of 1 mL/min using helium as carrier gas. Initial oven temperature was held at 60° C. for 1 min, increased to 140° C. at 25 C/min, to 250° C. at 5° C./min, and then increased to 300° C. at 10° C./min.

The carbohydrate composition of crude extract is reported in Table I, the carbohydrate composition of Fraction 3 is reported in Table II, below.

TABLE I
Carbohydrate compositions of crude Reishi extract

| Sugar components | Percentage (%) |
|---|---|
| D-Glucose | 58.0 |
| D-Mannose | 15.5 |
| L-Fucose | 9.7 |
| D-Galactose | 9.3 |
| D-Xylose | 5.4 |
| D-GlcNAc | 1.0 |
| L-Rhamnose | 0.5 |

TABLE II
Carbohydrate compositions of Fraction 3

| Sugar components | Percentage (%) |
|---|---|
| D-Glucose | 58.1 |
| D-Mannose | 15.1 |
| L-Fucose | 7.1 |
| D-Galactose | 13.5 |
| D-Xylose | 3.1 |
| D-GlcNAc | 1.3 |
| L-Rhamnose | 0.7 |

High-pH anion-exchange chromatography with pulsed amperometric detection (HPAEC/PAD) analysis, confirmed that F3 includes a glycoprotein or polysaccharide comprising fucose residues.

Also H2SO4/phenol analysis showed that overall polysaccharides concentration in F3 (85%) is higher than crude extract (60%).

### Amino Acid Composition Analysis

The analysis was carried out based on a well-established method (Spachman et al 1958; Lo et al 1990). A sample of crude Reishi extract (6 mg) was dissolved in 1 mL solution of 6 M HCl and TFA (4/1), and heated at 140° C. for 3 h. The mixture was concentrated to give a dry residue and dissolved in 100 μL citrate buffer. A small aliquot (4 μL) was withdrawn and subjected to composition analysis by amino acid analyzer (Jeol JLC-6AH).

The resulting amino acid composition of Reishi Crude extract is shown in Table III below.

TABLE III
Amino acid analysis of Reishi extract

| Amino acid | Relative abundance |
|---|---|
| Asp | 117 |
| Thr | 66 |
| Ser | 54 |
| Glu | 120 |
| Pro | 60 |
| Gly | 108 |
| Ala | 100 |
| Val | 61 |
| Mel | 6 |
| Ile | 36 |
| Leu | 55 |
| Tyr | 16 |
| Phe | 28 |
| His | 12 |
| Lys | 21 |
| Arg | 22 |

Analysis directed to investigate protein concentration in Reishi crude extract and in Fraction 3 showed a F3 (˜10%), crude extract (˜20%) in Lowey method with BSA as a standard.

Further indications concerning F3 composition, differences with composition of crude Reishi extract and procedures to obtain F3 can be found in Chen et al. 2004 herein incorporated in its entirety.

### Alternative Preparation of Reishi Extract Fraction 3

A crude G. Lucidum PS extract prepared via alkaline extraction with 0.1 N of NaOH, followed by neutralization and ethanol precipitation, was obtained from Pharmanex (CA). The crude G. Lucidum extract (100 g) was dissolved in 3 L of double-distilled H2O and stirred at 4° C. for 24 h. The solution was centrifuged (16,000 g) at 4° C. for 1 h, and the supernatant was concentrated at 35° C. The slurry product was then lyophilized to obtain 70 g of water-soluble dark brown G. Lucidum extract. The extract (2.5 g) was fractionated on Sephacryl S-500 column (95 2.6 cm) with 0.1 N of Tris buffer (pH 7.0) as the eluent. The flow rate was set at 0.6 ml/min, and fractions were collected with 7.5 ml per tube. Five fractions were collected, and each was dialyzed to remove excessive salts and lyophilized to give fractions 1-5; each fraction was characterized, as described above. The fucose-containing glycoprotein fraction (20˜30% yield), i.e., Fraction 3 or F3, was isolated.

To avoid LPS contamination the crude G. lucidum materials and PS extracts were prepared, from growth to harvest, as GMP grade from Pharmanex and the possible bacterial contamination was carefully monitored to meet the United States Food and Drug Administration standard. The reagents and utensils for preparation of F3 were either endotoxin-free grade or washed with PBS containing 50 μg/ml polymyxin B (PMB), then rinsed with PBS. F3 contained <1 ng of LPS/25 μg, as measured by LAL assay (Sigma-Aldrich). In addition, certain reagents were routinely checked by LAL for examination of LPS contamination.

An additional procedure was performed as described in Wang et al 2002 herein incorporated by reference in its entirety. According to embodiments, a modified version of the procedure described in Wang et al. comprises direct centrifugation, isolated polysaccharide from water soluble Reishi sample which showed components as well as F3.

The procedure as below: The water soluble polysaccharide from crude powder of Reishi 1 g was centrifuged (5000 r.p.m., 2800 g) at 4° C. for 1 h to separate polysaccharide by centrifugal filter with MWCO: 100K, the polysaccharide fraction was collected and lyophilized to give F3 (F3>100K) 172 mg (17%). This portion of polysaccharide shown familiar HPLC profile with F3 and its bio-function assay was analyzed, such as effect of proliferation and cytokines release to mice splenocyte, as well as previously F3 function.

### Preparation and Purification of Reishi Extracts F3-F3G1 FRG2, F3 GH1, and F3 GH2

Crude Reishi extract (prepared via alkaline extraction (0.1 N NaOH), neutralization and ethanol precipitation) was obtained from Pharmanex Co., (CA, USA). All the chemicals and reagents were from Sigma Co., (St. Louis, Mo., USA) unless indicated.

Crude Reishi extract (100 g) was dissolved in 3 L of double distilled water, stirred at 4° C. for 24 h, and centrifuged for 1 h to remove the insoluble. The resulting solution was concentrated at 35° C. to give a small volume and lyophilized to generate 70 g powder of dark-brown color, 2.5 g of which were dissolved in a small volume of Tris buffer (pH 7.0, 0.1 N), and purified by gel filtration chromatography using a Sephacryl S-500 column (95×2.6 cm) with 0.1 N Tris buffer (pH 7.0) as the eluent. The flow rate was set at 0.6 mL/min, and 7.5 mL per tube was collected. After the chromatography, each fraction was subjected to anthrone analysis or the phenol-sulfuric acid method as described above, to detect sugar components. Five fractions were collected (fractions 1-5), each dialyzed to remove excessive salt and lyophilized, to give 450 mg of each fraction and in particular of F3.

F3 was further subjected to a column of Diaion-WA30 anion exchanger (Cl-form, 40×3.5 cm) eluted with 0.2 and 0.8 M NaCl at a flow rate of 0.5 mL/min, and two fractions were designated as F3G1 (11% yield based on F3) and F3G2 (10% yield based on F3), respectively, as shown in FIG. 14. Another fraction (F3G3, 11% yield based on F3) was generated when the column was further eluted with 2 M NaOH.

The carbohydrate composition of the F3G1, F3G2 and F3G3 was determined by anthrone colorimetric method and TMS method. The results are shown in Table IV below.

TABLE IV
Carbohydrate compositions of F3, F3G1, F3G2, and F3G3

| | Percentage (%) | | | | | | |
|---|---|---|---|---|---|---|---|
| | L- | D- | | | | | |
| | Fuc | Xyl | D-Man | D-Gal | D-GlcNAc | D-Glc | Unknown |
| F3 | 7.1 | 3.1 | 15.1 | 13.5 | 1.20 | 58.1 | 1.90 |
| F3G1 | 8.0 | 5.7 | 10.2 | 12.6 | 0.25 | 63.2 | 0.05 |
| F3G2 | 6.2 | 4.5 | 18.3 | 5.3 | 0.78 | 64.9 | 0.02 |
| F3G3 | 8.4 | 7.2 | 14.5 | 2.9 | 1.18 | 65.7 | 0.12 |

The results show that both Fraction 3 and the subfractions F3G1, F3G2 and F3G3 comprise glucose and mannose as major components together with smaller amounts of other sugars, including fucose N-acetylglucosamine, xylose and rhamnose, The percentage of galactose is apparently less in F3G2 and F3G3 than in other fractions.

Gel-filtration chromatography of F3G2 was carried out on a TSK HW-75 column (130×2.6 cm) eluted with double distilled water at a flow rate of 0.5 mL/min. Two fractions were collected; F3G2H1 (19% yield based on F3G2) and F3G2H2 (69% yield based on F3G2), as shown in FIG. 15.

Further indications concerning the composition of F3G1, F3G2, F3G3, F3G2H1 and F3G2H2 and procedures to obtain the Subfractions can be found in Chen et al. 2004, herein incorporated in its entirety.

In one implementation, F3 (or subfractions thereof that comprise fucose-containing glycoprotein(s)) is administered to a mammal or a bird to prevent or treat infection with influenza virus, including but not limited to influenza type A virus strains. Type A strains include H1N1, H2N2, H3N2, H5N1, H7N7, H1N2, H9N2, H7N2, H7N3, and H10N7. The inventors of this disclosure discovered that treatment of mice with F3 of Reishi shortly after infection by a lethal dose of influenza virus can prolong their survival. Additionally, the inventors discovered that when F3 treatment of mice is started before challenge with a lethal dose of influenza virus, the incidence of lethality is drastically reduced.

When administered to a mammal (e.g., a human or a mouse) or a bird, therapeutically effective amounts of F3 (or subfractions thereof that comprise fucose-containing glycoprotein(s)) may be in the form of a pharmaceutical compositions which may also comprise one or more pharmaceutically acceptable carriers, diluents, or excipients. The administration of the pharmaceutical composition for the prevention or treatment of viral infections, such as influenza infections, may occur via any route known in the art, for example via topical administration, via enteral administration (e.g. orally or rectally), or via parenteral administration (e.g. intravenously, intra-muscularly, or subcutaneously).

### EXAMPLES

The following examples are non-limiting and are provided solely to further illustrate the methods and compositions of the disclosure.

### Example 1

### Use of F3 to Treat H1N1 Influenza Virus Infection

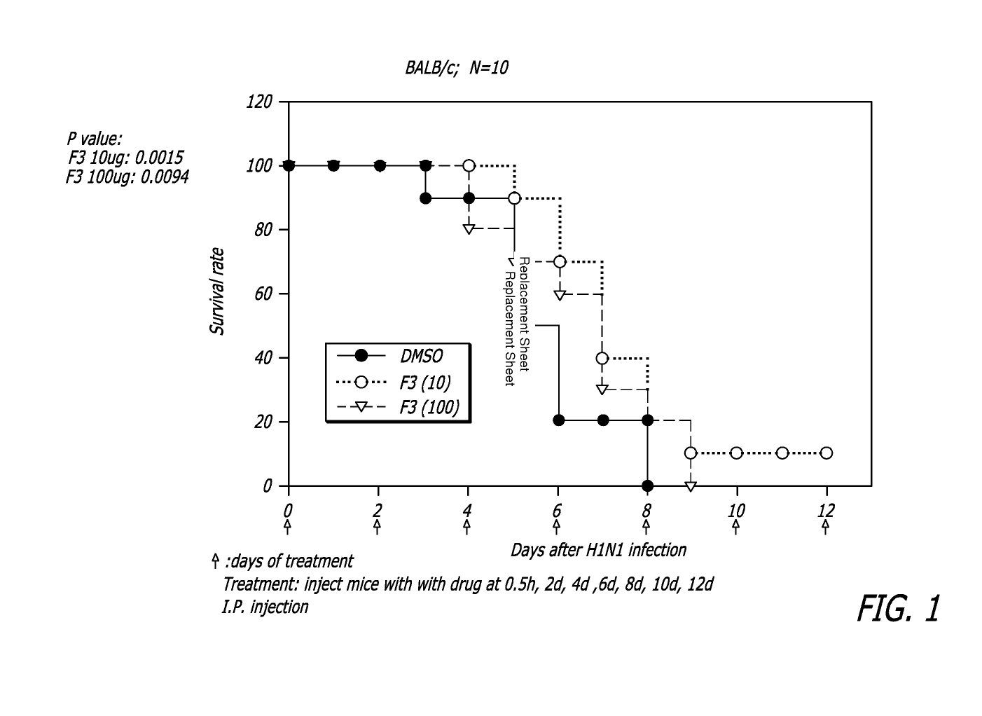

Thirty minutes after infection with a lethal dose of H1N1, BALB/c mice were treated with 10 μg or 100 μg of F3 (administered via intra-peritoneal injection). F3 treatment was then repeated every other day for 12 days or until death. As shown in FIG. 1, 10/10 control mice died by day 8. Survival curve analysis showed that mice treated with either 10 or 100 μg F3 had significantly prolonged survival with p=0.0015 and 0.0094, respectively. One of the 10 mice treated with 10 μg F3 survived the infection.

### Example 2

### Pretreatment with F3 Prior to Infection with H1N1 Influenza Virus

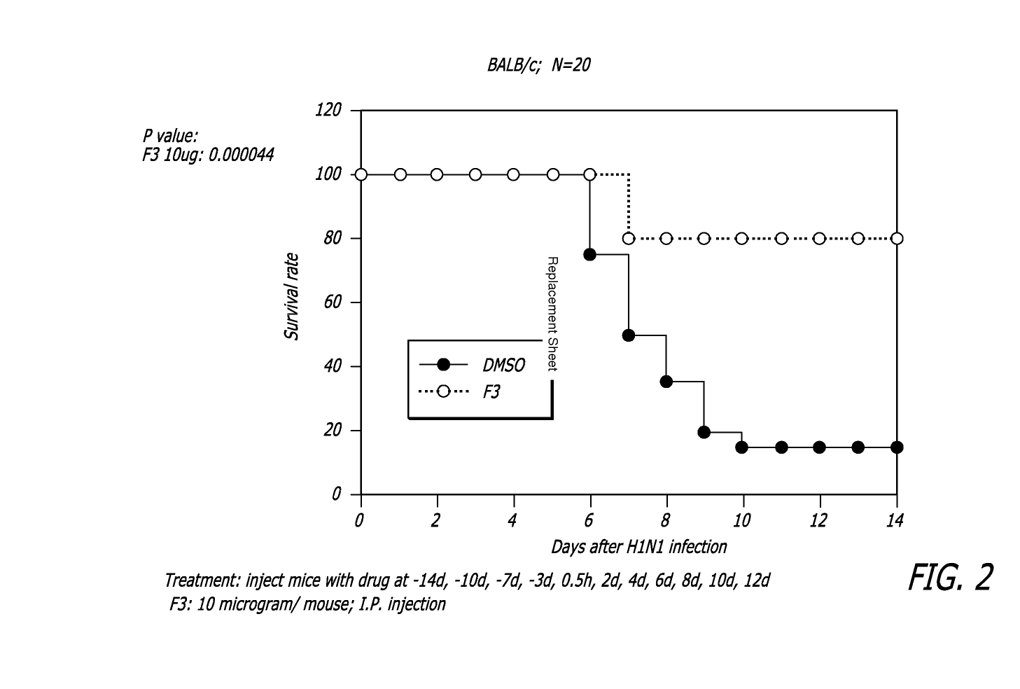

In another study, BALB/c mice were pretreated twice per week for two weeks with 10 μg of F3 (administered via intra-peritoneal injection) and then challenged with 10 LD50 of H1N1. F3 treatment was then continued every other day for 12 days or until death. As shown in FIG. 2, only 3/20 (15%) of the mice in the control group survived, whereas 16/20 (80%) F3 treated mice survived (p=0.000044).

### Example 3

### Pretreatment with Various Dosages of F3 Prior to Infection with Influenza Virus

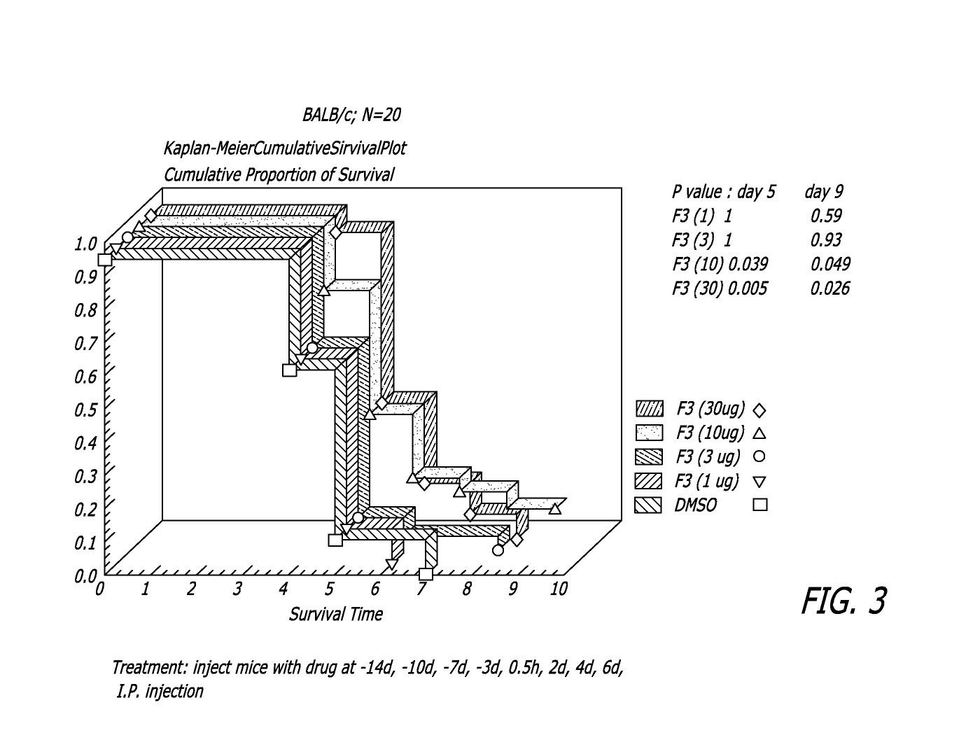

BALB/c mice were pretreated twice per week for two weeks with 1-30 μg of F3 (administered via intra-peritoneal injection) and then challenged with 10 LD50 of H1N1. F3 treatment was then continued every other day for 6 days or until death. As shown in FIG. 3, 100% of the mice in the control group died by day 7, with median survival of 5 days, whereas mice treated with 10 and 30 μg had significantly prolonged survival, with median survival of 6 days (p=0.049, and 0.026, respectively). Four of the 20 mice treated with 10 μg F3 survived the infection.

### Example 4

### Pretreatment with Various Dosages of F3 Prior to Infection with Different Viral Titers of Influenza Virus

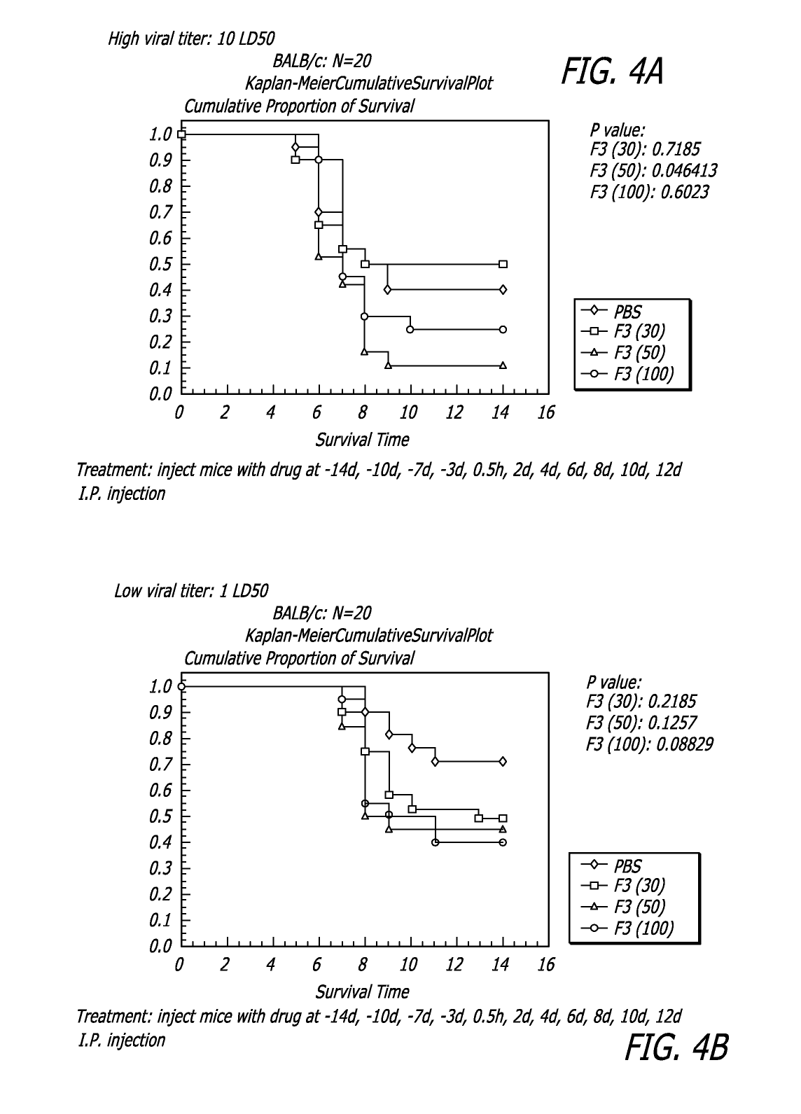

BALB/c mice were pretreated twice per week for two weeks with 30-100 μg of F3 (administered via intra-peritoneal injection) and then challenged with 10 LD50 or 1 LD50 of H1N1 (FIGS. 4A, and 4B, respectively). F3 treatment was then continued every other day until death. As shown in FIG. 4A, 60% of the mice in the control group died by day 9, whereas mice treated with 30 and 100 μg had no significantly prolonged survival (p=0.7185, and 0.6023, respectively). However, mice treated with 50 μg appeared to be worse than control on survival rate (p=0.046). Ten, two, and five of the 20 mice treated with 10, 50, and 100 μg F3, respectively, survived the infection. We than repeated the same experiment with a lower virus titer at 1LD50. As shown in FIG. 4B, 30% of the mice in the control group died by day 11, whereas mice treated with 30, 50, and 100 μg had no significantly prolonged survival (p=0.2185, 0.1257, and 0.0882, respectively). Ten, ten, and nine of the 20 mice treated with 10, 50, and 100 μg F3, respectively, survived the infection.

### Example 5

### Pretreatment with Different Batch of F3 and 1-3Beta Glucan Prior to Infection with Influenza Virus

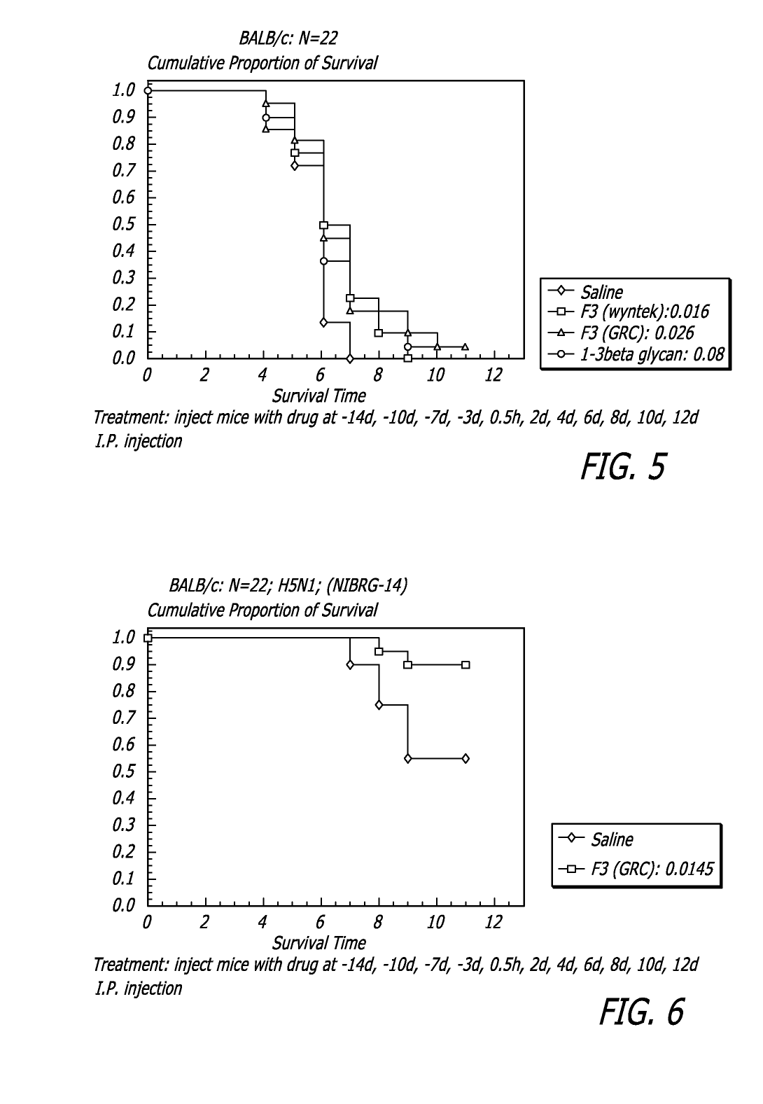

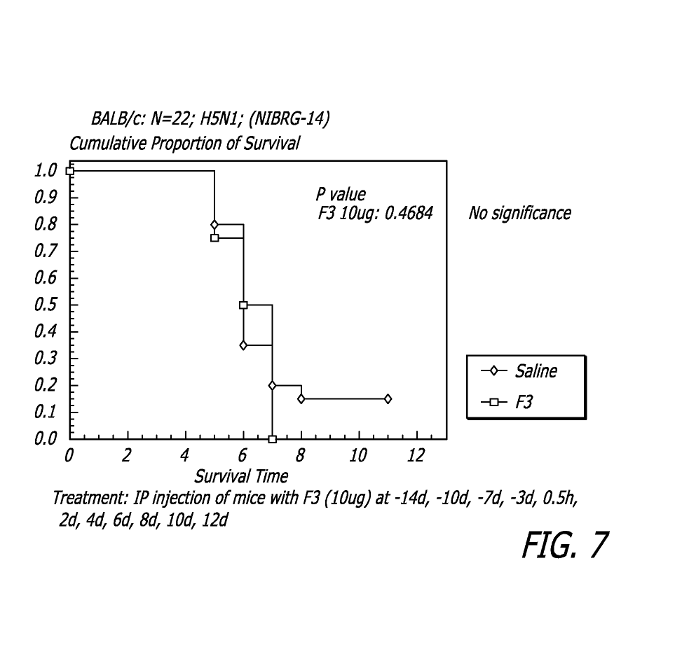

BALB/c mice were pretreated twice per week for two weeks with 10 μg F3 from Wyntek company, F3 from GRC, and the backbone of F3, 1-3 beta glucan (administered via intra-peritoneal injection), and then challenged with 10 LD50 of H1N1. F3 treatment was then continued every other day for 6 days or until death. As shown in FIG. 5, 100% of the mice in the control group died by day 7, whereas mice treated with 10 μg F3 from Wyntek Inc. and GRC had significantly prolonged survival, (p=0.016, and 0.026, respectively). Although one of the 20 mice treated with 10 μg 1-3 beta glucan survived the infection, the survival of the entire group did not differ significantly from the control group (p=0.08).

### Example 6

### Pretreatment with F3 Prior to Infection with H5N1 Influenza Virus

BALB/c mice were pretreated twice per week for two weeks with 10 μg of F3 (administered via intra-peritoneal injection) and then challenged with 20 LD50 of H5N1 (NIBRG-14). F3 treatment was then continued every other day for 12 days or until death. As shown in FIG. 6, only 13/22 (59%) of the mice in the control group survived, whereas 20/22 (90%) F3 treated mice survived the infection (p=0.0145).

### Example 7

### Pretreatment with F3 Prior to Infection with Lower Titer of H5N1 Influenza Virus

In another study, BALB/c mice were pretreated twice per week for two weeks with 10 μg of F3 (administered via intra-peritoneal injection) and then challenged with 2 LD50 of H5N1 (NIBRG-14). F3 treatment was then continued every other day for 12 days or until death. As shown in FIG. 6, 4/22 (18%) of the mice in the control group survived, whereas 22/22 (100%) F3 treated mice died by day 7 and shown no statistically difference in comparison to control (p=0.4684).

### Example 8

### Kinetics of Serum Cytokine/Chemokine Profile in Response to F3

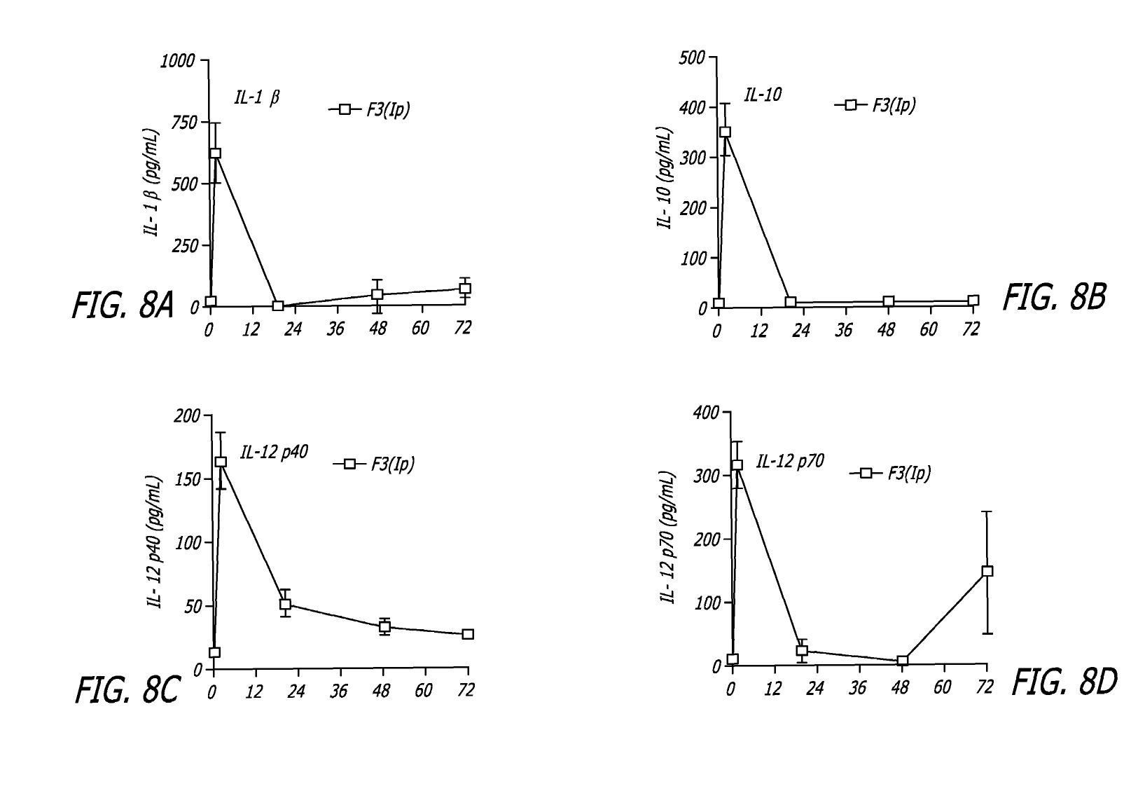

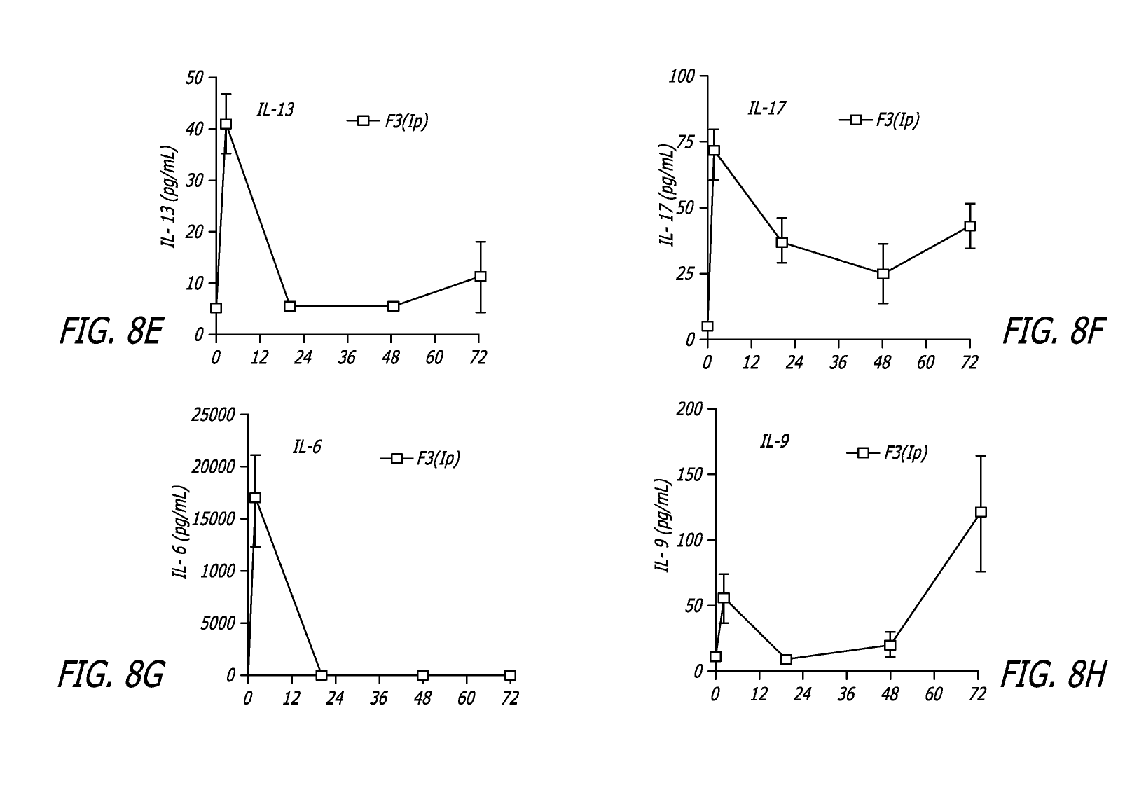

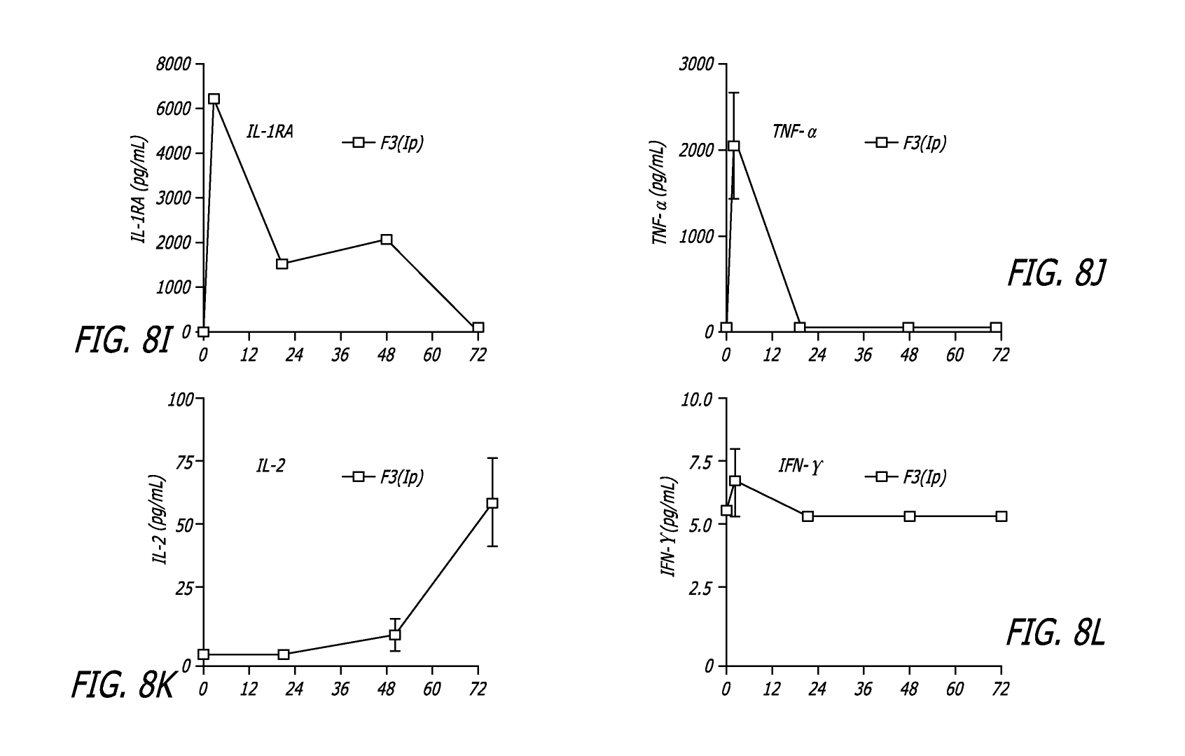

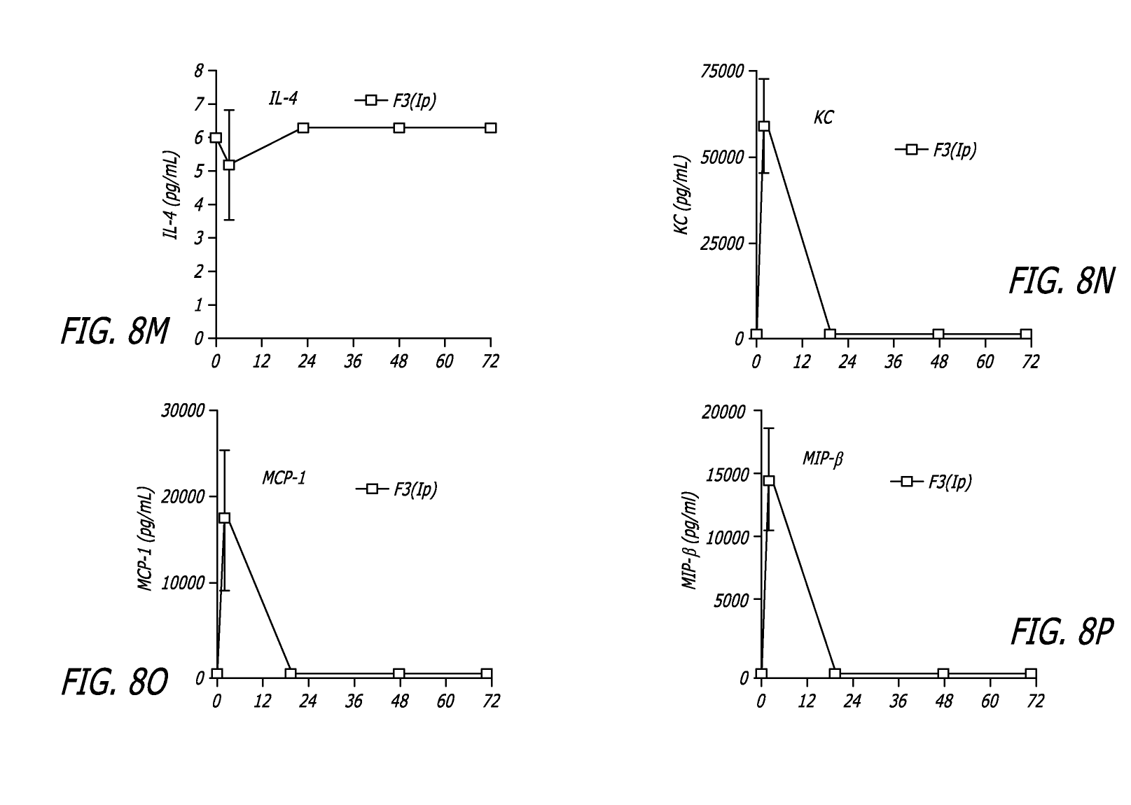

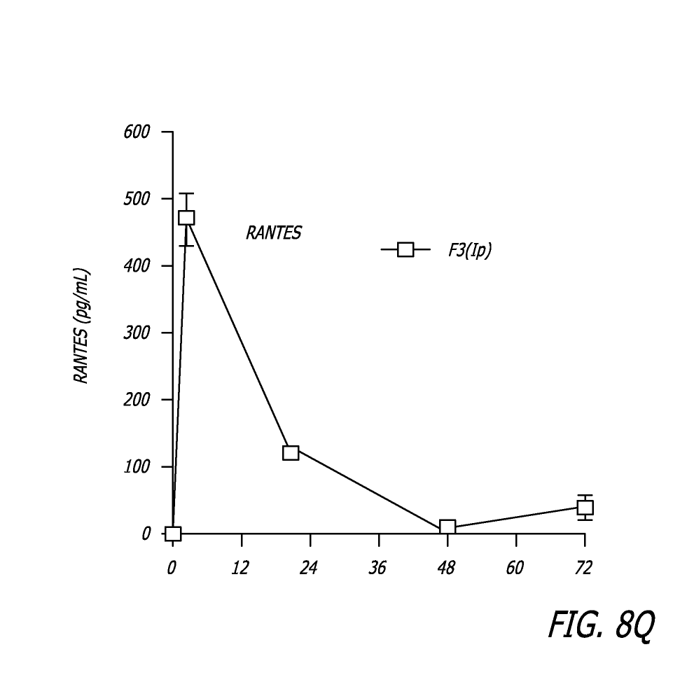

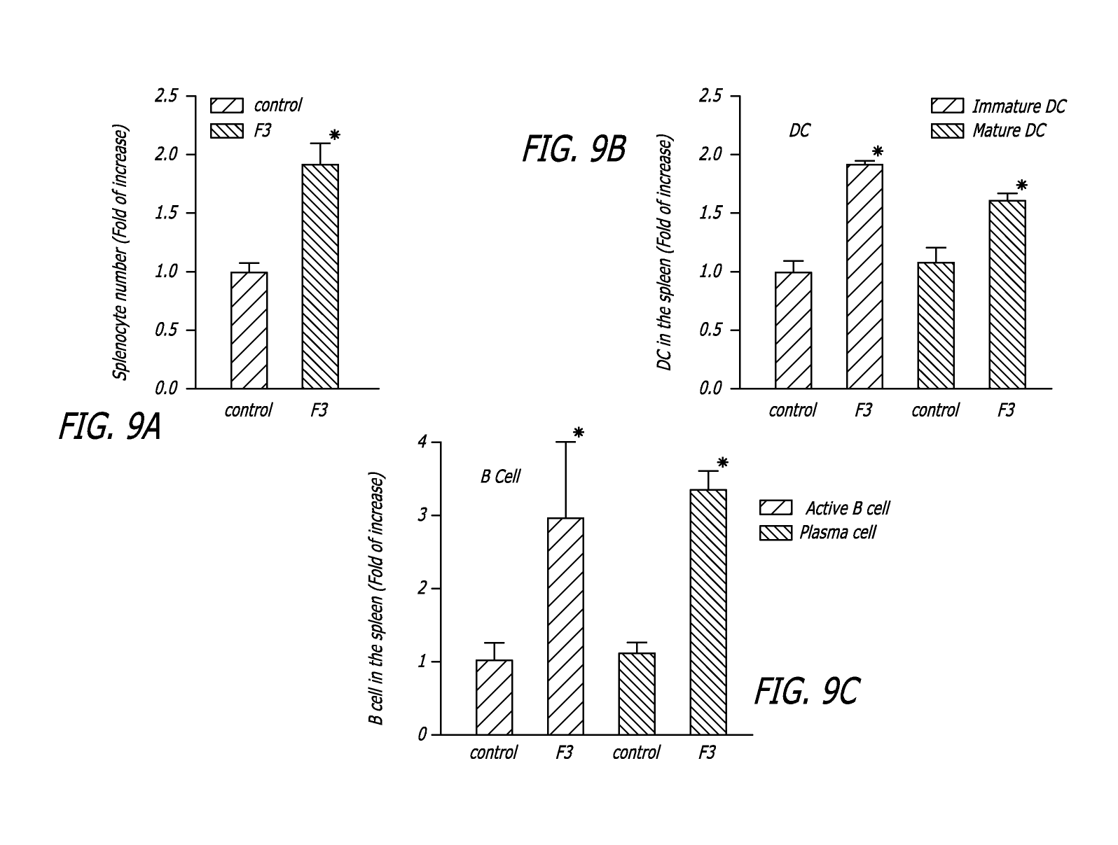

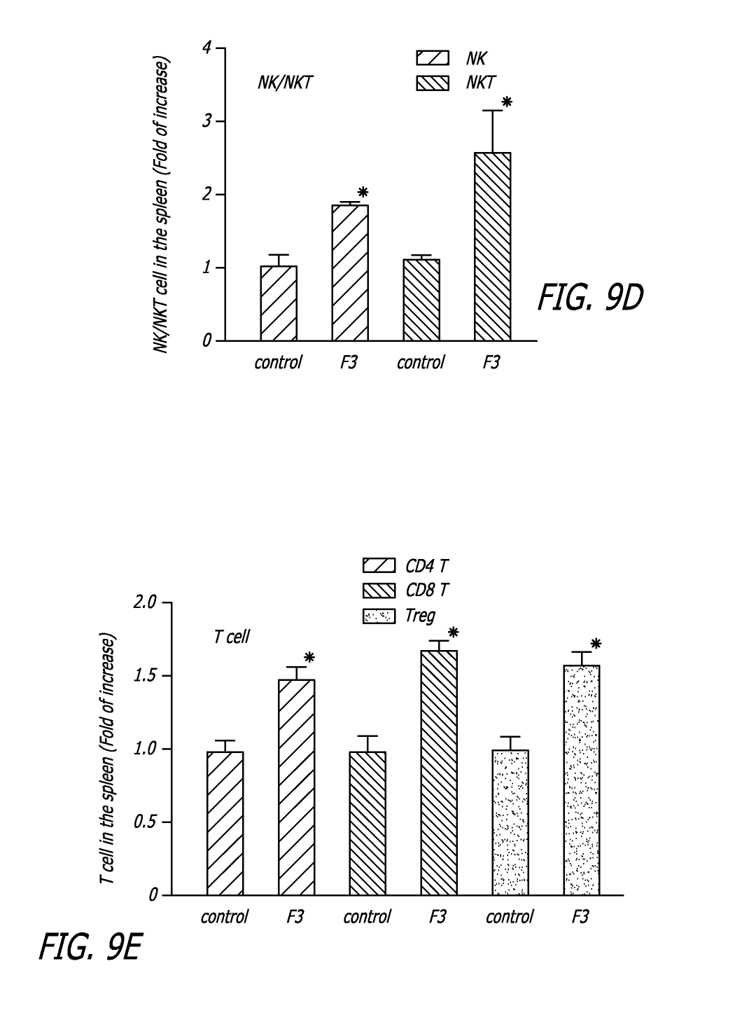

BALB/c mouse serum samples (N=3) were collected at 0, 2, 18, 48, and 72 hr after intra-peritoneal injection of 10 μg F3. The serum concentrations of 21 cytokines/chemokines were measured by Beadlyte Mouse 21-plex Cytokine Detection System (cat #48-012, Upstate, NY), and read by a Luminex 100™ system. IL-1RA is measured by another ELISA kit. As shown in FIG. 8, most cytokines/chemokines, such as IL-1β (A), IL-10 (B), IL-12 p40 (C), IL-12 p70 (D), IL-13 (E), IL-17 (F), IL-6 (G), IL-9 (H), IL-1RA (I), TNF-α (J), KC (N), MCP-1 (0), MIP-β (P), RANTES (Q), have similar kinetics that reached their peaks at 2 hr and declined at 18 hr post-injection. In comparison, IL-2 (K) increased at 48 hr and reached its peak at 72 hr post-injection. However, IFN-γ (L) and IL-4 (M) as well as IL-1α, IL3, IL-5, GM-CSF, and VEGF (data not shown) had no significant increase during 72 hr.

### Example 9

### Expansion/Activation of Splenocytes after Single Injection of F3

Spleens from BALB/c mice (N=3) were harvested 72 h after injection of F3 intraperitoneally. The increase of the total number of splenic nucleated cells was shown as (A). The induction of the subpopulation of immune cells by F3, including mature dendritic cells (CD11C+/CD80+/CD86+), activated B cells (CD45R+/CD23+/CD69+) and plasma cell (CD45R/CD138), activated NK cells (U5A2-13Ag+/CD3−/CD69+), activated NKT cells (U5A2-13Ag+/CD3+/CD69+), CD4 (CD3+/CD4+/CD8−), CD8 (CD3+/CD4−/CD8+), and Treg (CD4+/CD25+/FoxP3+) were shown in (B-E, respectively). Quantitative data are shown as the mean ±s.d. *, p<0.05, compared with saline control.

### Example 10

### Expression of Granzyme B in Splenocytes after Injection of F3

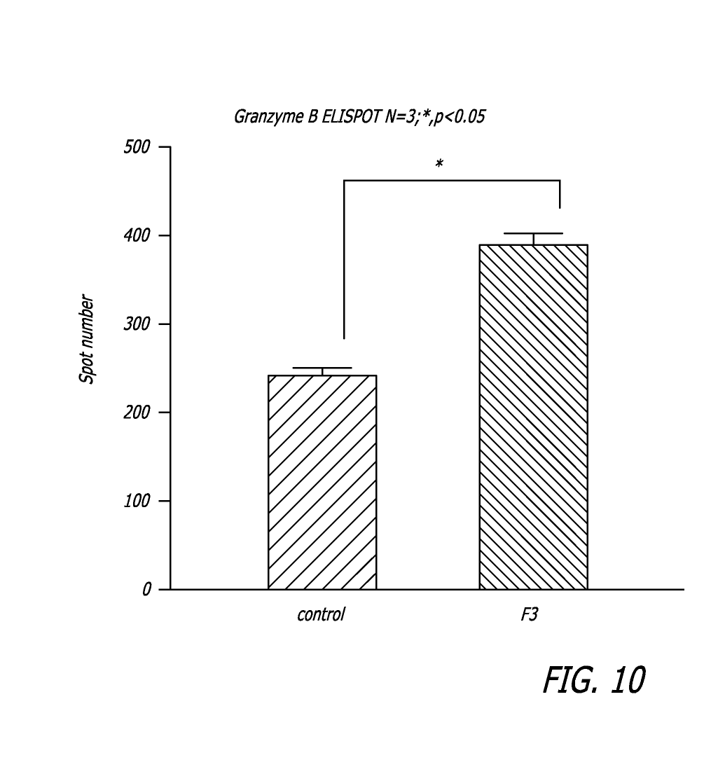

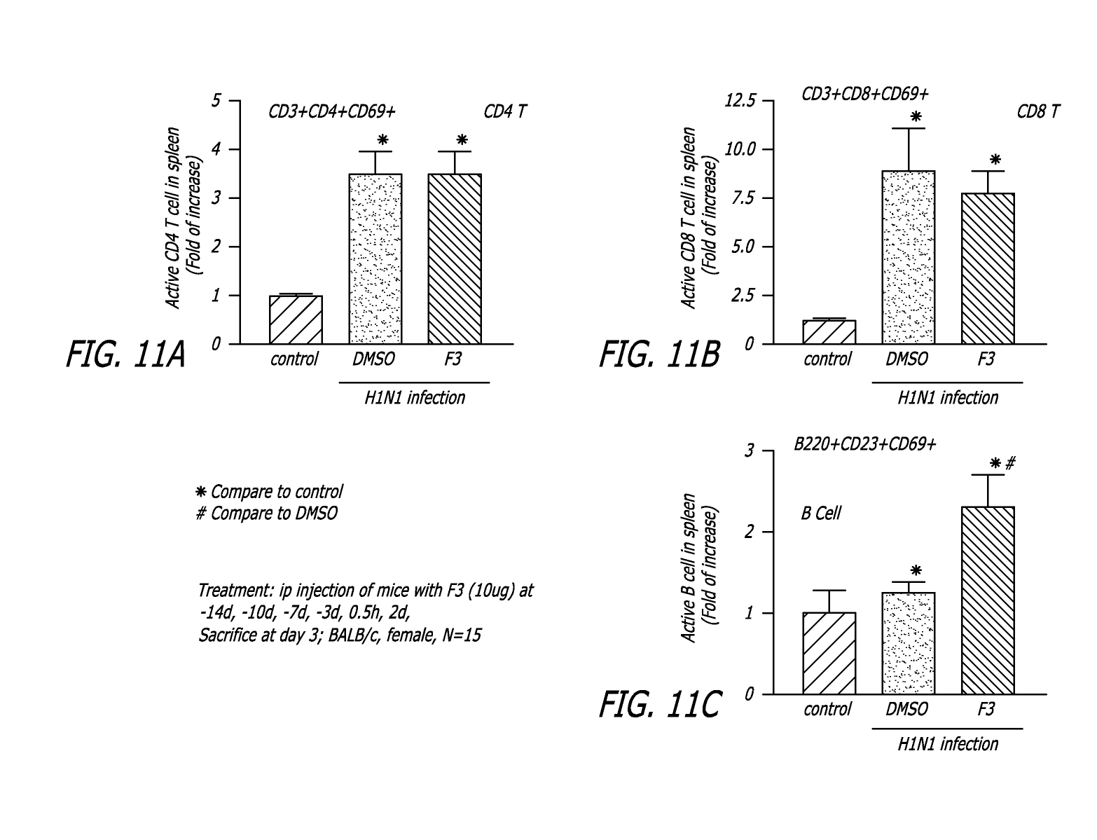

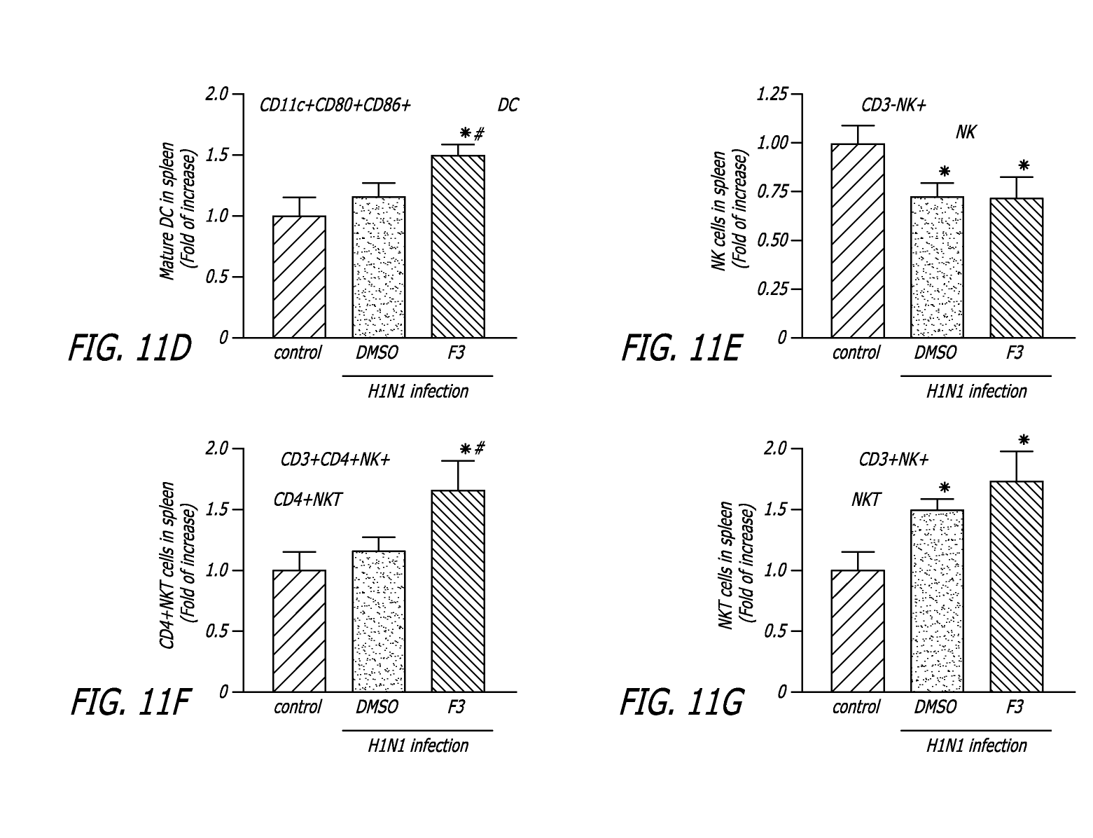

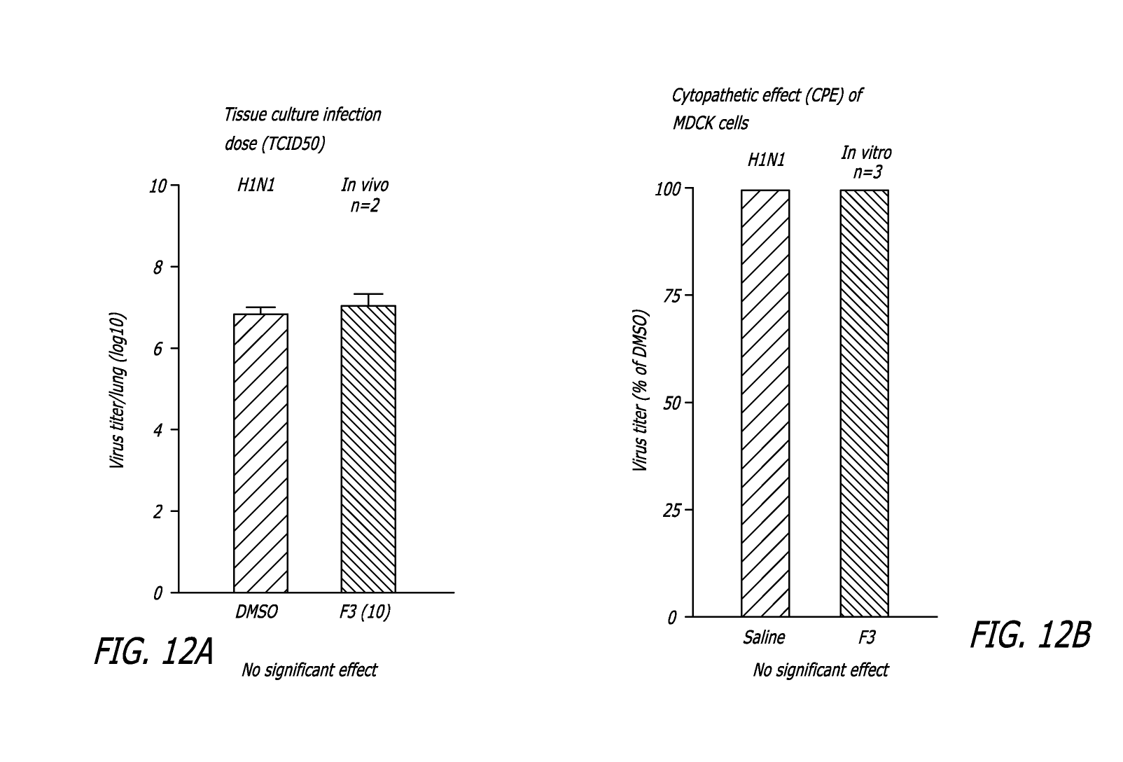

Spleens from BALB/c mice (N=3) were harvested 72 h after injection of F3 intraperitoneally, and then cultured in vitro for 48 hr to determine the production of Granzyme B by ELISPOT assay. Briefly, 5×104 splenocytes were cultured in 96-well culture plate pre-coated with anti-Granzyme B Ab, and then treated with 10 ug/ml F3 or control saline and incubated for another 48 hr. After that, cells were washed and the number of Granzyme B secreting-cells was detected as the color spots, using Ab and substrates, following manufacture's protocol. Data were obtained from six repeats and shown as the mean ±s.d. *, p<0.05, compared with saline control. As shown in FIG. 10, F3 increased the number of granzyeme B-secreting cells in spleen.

### Example 11

### Expansion/Activation of Splenocytes after Influenza Virus Infection and F3 Treatment

BALB/c mice were pretreated twice per week for two weeks with 10 μg of F3 (administered via intra-peritoneal injection) and then challenged with 10 LD50 of H1N1. Spleens from BALB/c mice (N=3) were harvested 72 h after virus infection. Among the immune effector cells, activated CD4 (CD3+/CD4+/CD69+), CD8 (CD3+//CD8+/CD69+) T cells increased after virus infection, while treatment of F3 did not show significant difference from the control (A and B, respectively). However, an increase in activated B cells (CD45R+/CD23+/CD69+), dendritic cells (CD11C+/CD80+/CD86+), and CD4+NKT cells (U5A2-13Ag+/CD3+/CD4+) were noted only in F3 treated mice after virus infection (C, D, and F, respectively). The number of NK cells (U5A2-13Ag+/CD3−) significantly decreased after virus infection in both F3 and control mice (E), whereas NKT cells (U5A2-13Ag+/CD3+) significantly increased after virus infection in both F3 and control subjects Quantitative data are shown as the mean ±s.d. *, p<0.05, compared with normal control; #, p<0.05, compared with infected control.

### Example 12

### Effect of F3 on Entry/Replication of H1N1 Virus In Vivo/In Vitro

BALB/c mice were pretreated twice per week for two weeks with 10 μg of F3 (administered via intra-peritoneal injection) and then challenged with 10 LD50 of H1N1. Lungs from infected BALB/c mice (N=2) were harvested 72 h post-infection for assessment of the surviving virus titer. The total virus in each lung extract were individually calculated as tissue culture infection dose (TCID50) and converted the data to virus titer/lung as shown in (A). In this experiment, it appears that F3 did not inhibit the virus replications in vivo.

In order to determine whether F3 directly interferes with the ability of H1N1 virus to infect target cells, MDCK cells (2×105) were cultured in 96-wells plate and pretreated with F3 (10 μg/ml) (B) or saline for 2 hrs. After that, MDCK cells were infected by H1N1 virus at the titer of 1×TCID50, and then cultured for another 48 hrs. The cytopathetic effects (CPE) on MDCK cells were observed by microscope. In comparison to control, there was no significant direct effect of F3 on virus entry/replication.

### Pharmaceutical Compositions

According to another aspect, the fucose-containing glycoprotein fraction can be included in a pharmaceutical composition together with additional active agents, carriers, vehicles, excipients, or auxiliary agents identifiable by a person skilled in the art upon reading of the present disclosure.

The pharmaceutical compositions comprise at least one pharmaceutically acceptable carrier. In such pharmaceutical compositions, the fucose-containing glycoprotein forms the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Subject as used herein refers to humans and non-human primates (e.g. guerilla, macaque, marmoset), livestock animals (e.g. sheep, cow, horse, donkey, pig), companion animals (e.g. dog, cat), laboratory test animals (e.g. mouse, rabbit, rat, guinea pig, hamster), captive wild animals (e.g. fox, deer), and any other organisms who can benefit from the agents of the present disclosure. There is no limitation on the type of animal that could benefit from the presently described agents. A subject regardless of whether it is a human or non-human organism may be referred to as a patient, individual, animal, host, or recipient.

Pharmaceutical compositions suitable for an injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. According to embodiments, isotonic agents, for example, sugars, polyalcohols such as manitol, sorbitol, or sodium chloride in the composition are added. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preparation are prepared by vacuum drying or freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

According to embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of the active compound (i.e., an effective dosage) may range from about 0.001 to 30 mg/kg body weight. The skilled artisan will appreciate that certain factors can influence the dosage and timing required to effectively treat a subject, including but not limited to the severity of the disease or disorder, previous treatments, the general health or age of the subject, and other diseases present.

According to another aspect, one or more kits of parts can be envisioned by the person skilled in the art, the kits of parts to perform at least one of the methods herein disclosed, the kit of parts comprising two or more compositions, the compositions comprising alone or in combination an effective amount of fucose-containing glycoprotein fraction according to the at least one of the above mentioned methods.

The kits possibly include also compositions comprising active agents other than the fucose-containing glycoprotein fraction, identifiers of a biological event, or other compounds identifiable by a person skilled upon reading of the present disclosure. The term “identifier” refers to a molecule, metabolite or other compound, such as antibodies, DNA or RNA oligonucleotides, able to discover or determine the existence, presence, or fact of or otherwise detect a biological event under procedures identifiable by a person skilled in the art; exemplary identifiers are antibodies, exemplary procedures are western blot, nitrite assay and RT-PCR, as described in the Examples. Exemplary biological events are cytokine expression or other immunomodulating events; an exemplary active agent other than the fucose-containing glycoprotein fraction is LPS.

The kit can also comprise at least one composition comprising an effective amount of fucose-containing glycoprotein fraction or a cell line. The compositions and the cell line of the kits of parts to be used to perform the at least one method herein disclosed according to procedure identifiable by a person skilled in the art.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

### REFERENCES

The following references are each incorporated herein by reference in their entirety. The citation of these references is not an admission that any of them forms part of the body of the prior art in any country.

1. A composition comprising:
a fucose-containing glycoprotein extract of Ganoderma lucidum;
wherein the extract is used to treat an organism having a virus.

2. The composition of claim 1, wherein the virus comprises an influenza virus.

3. The composition of claim 2, wherein the influenza virus comprises type A influenza.

4. The composition of claim 1, wherein the organism is a human.

5. The composition of claim 1, wherein the fucose-containing glycoprotein extract has a light absorbance of about 1.8 at O.D. 625

6. A composition comprising:
at least a fucose-containing glycoprotein fraction of Ganoderma lucidum,
wherein at least the fucose-containing glycoprotein fraction is used for the treatment of virus infections in an organism.

7. The composition of claim 6, further comprising a pharmaceutically acceptable carrier.

8. The method of claim 6, wherein the virus comprises an influenza virus.

9. The method of claim 8, wherein the influenza virus comprises type A influenza.

10. A method comprising:
administering to an organism a pharmacologically effective amount of a fucose-containing glycoprotein extract of Ganoderma lucidum to treat virus infections in an organism.

11. The method of claim 11, wherein administration of the fucose-containing glycoprotein extract occurs at least prior to the infection of the organism by the virus.

12. The method of claim 11, wherein the virus is an influenza virus.

13. The method of claim 12, wherein the influenza virus is a type A influenza virus.

14. A kit comprising:
at least a fucose-containing glycoprotein extract of Ganoderma lucidum.
